(19) United States
(12) Patent Application Publication
(10) Pub. No.: US 2018/0344583 A1
(43) Pub. Date: Dec. 6, 2018

(54) Powdered Nail Preparations and Methods of Application

(71) Applicant: Cali Chem, Inc., Garden Grove, CA (US)

(72) Inventor: Amy Doan, Garden Grove, CA (US)

(73) Assignee: Cali Chem, Inc., Garden Grove, CA (US)

(21) Appl. No.: 16/000,636

(22) Filed: Jun. 5, 2018

Related U.S. Application Data
(60) Provisional application No. 62/515,441, filed on Jun. 5, 2017.

(51) Int. Cl. A61K 8/02; A61K 8/87; A61K 8/81; A61K 8/35; A61K 8/55; A61K 8/34; A61Q 3/02

(52) U.S. Cl. CPC A61K 8/022; A61K 8/87; A61K 8/8147; A61K 8/35; A61K 2800/43; A61K 8/347; A61Q 3/02; A61K 2800/95; A61K 2800/81; A61K 8/55

(57) ABSTRACT

Methods and processes for applying powder coat nail preparations without the need for the use of glues are provided. The process includes the combination of a base color gel and a powder layer, and curing that combination to build up a plurality of layers on the nail. The layered gel/powder coating may be finished with a top gel coat material, followed by a further curing process. The process may also include using a preparation material prior to applying the base gel and powder coatings. The process may also include buffing and finishing the nail prior to and after applying the top gel coat material.

### CROSS-REFERENCE TO RELATED APPLICATIONS

The current application claims priority to U.S. Provisional Application No. 62/515,441, filed Jun. 5, 2017, the disclosure of which is incorporated herein by reference.

### FIELD OF THE INVENTION

The present invention relates to powder nail preparations and methods of applying and finishing the same.

### BACKGROUND OF THE INVENTION

Light or radiation curable preparations for use on nails have been known for many years. In these formulations ultraviolet (UV) lamps are used to seal the coated nail (a process called ‘curing’). These radiation curable preparations are favored over traditional acrylic nail preparations, because they can last for a significantly longer period of time, in some cases weeks longer. Partially as a result any different formulations have been described, based on a variety of radiation curable materials, such as, for example, aliphatic hydrocarbon urethane diacrylate and methacrylates (such as are described in U.S. Pat. No. 4,704,303), acrylate urethane oligomers that are crosslinked on curing with UV light (such as are described in U.S. Pat. No. 4,682,612), and more recently bisphenol A diglycidyl methacrylates (‘BISGMA’) (such as are described in U.S. Pat. No. 6,803,394).

Despite the number of different types of formulations, to date the application of such UV curable nail preparations typically require a time-consuming multi-step process. For example, most preparations require not only the cleaning of the underlying nail (whether natural or artificial) by filing, buffing, washing, etc., but additionally requires the application of a base coat to prepare the nail for the application of the coating or artificial nail, and ensure good binding of the coating or artificial nail to the nail substrate. Then, after the coating or artificial nail has been applied and UV cured, a topcoat is applied to provide a hardened protective outer layer to protect the colored nail from damage and wear. Such a process can take thirty minutes or more to apply, set and dry making it inconvenient for the wearer and nail professional.

An older alternative to these processes is the powder coat or acrylic dip system. These systems use an activated glue in combination with an acrylic powder to form the nail coating. Although these nail coating materials do offer some advantages over radiation curable nail materials, including ease of application and the lack of a requirement to use fans, acrylic brushes, French cutting tools, and other implements needed to apply radiation curable nail materials, they have fallen out of favor because the glues used are extremely toxic and removal can be problematic, requiring an extended soak-off period. Accordingly, a need exists for processes of improving the application of powder coat nail materials.

### SUMMARY OF THE INVENTION

Powder coat nail preparations, and methods of their application are described.

In some embodiments the invention is directed to a radiation compatible powder nail coating system including:
a base radiation curable nail material comprising at least a polymerizable material and a photoinitiator;
at least one base and one colored powderized coating material, each comprising at least a polymerizable monomer, and one of which further comprising a pigment material;
a top-coat radiation curable nail material comprising a polymerizable material and a photoinitiator; and
a radiative source.

In other embodiments, the radiation compatible powder nail coating system includes a solvent-based resin preparation material.

In still other embodiments, the base radiation curable nail material and the top-coat radiation curable material comprise at least one material selected from the group consisting of acrylates, (meth)acrylates, urethane(meth)acrylates, aliphatic polyols, aromatic polyols, polyester polyols, polyether polyols, aliphatic polyurethanes, aromatic polyurethanes, polyester polyurethanes, polyether diisocyanates, di-Hema trimethylhexyl dicarbamate), 2-hydroxyethyl methacrylate, hydroxypropyl methacrylate, and mixtures thereof.

In yet other embodiments, the base radiation curable nail material and the top-coat radiation curable material comprise at least one material selected from the group consisting of ketones, benzyl ketones, sulfanyl ketones, monomeric hydroxyl ketones, polymeric hydroxyl ketones, phosphinates, acyl phosphine oxides, metallocenes, benzophenone, hydroxycyclohexylphenylketone, trimethylbenzoylphosphine oxid), and mixtures thereof.

In still yet other embodiments, the radiation compatible coating system includes a material selected from the group of photoaccelerators, coupling agents, plasticizers, additional photoinitiators, colorants, solvents, dyes, preservatives, inhibitors, fillers, fibers, and adhesion promoting polymers

In still yet other embodiments, the base radiation curable nail material comprises from 55 to 85% by weight of one or more polymerizable material, 0.2 to 2% by weight of one or more photoinitiator, one or more inhibitor, and from 2 to 10% by weight of one or more solvent.

In still yet other embodiments, the polymerizable material comprises a mixture of polyurethane acrylate oligomer and 2-hydroxyethyl methacrylate, the photoinitator comprises one or more of hydroxycyclohexyl phenyl ketone and trimethylbenzoyl diphenylphosphine oxide, the inhibitor comprises one or more of hydroquinone and p-hydroxyanisole, and wherein the solvent is one or more of butyl acetate, ethyl acetate and isopropyl alcohol.

In still yet other embodiments, the top-coat radiation curable material comprises from 50 to 85% by weight of one or more polymerizable materials, 0.1 to 6% by weight of one or more photoinitiator, one or more inhibitor, and from 2 to 10% by weight of one or more solvent.

In still yet other embodiments, the powderized coating materials comprise one or more materials selected from the group of acrylates, (meth)acrylates, urethane(meth)acrylates, and polyether diisocyanates.

In still yet other embodiments, the radiation compatible coating system includes a preparatory base coat comprising a resin material, a polymerizable material and a solvent.

In still yet other embodiments, the resin material comprises from 20 to 30% by weight, the polymerizable material comprises from 20 to 30% by weight, and wherein the solvent comprises from 40 to 60% by weight.

In still yet other embodiments, the preparatory base coat comprises one or more material selected from the group consisting of bisphenol A glycidyl methacrylate (bis-GMA), (meth)acrylated monomers, urethane(meth)acrylates, polyurethanes, polyether diisocyanates, di-Hema trimethylhexyl dicarbamate), 2-hydroxyethyl methacrylate (HEMA) and hydroxypropyl methacrylate.

In still yet other embodiments, the preparatory base coat comprises 2,2-bis-(4-(2-hydroxy-3-methacryloxypropoxy)BIS-GMA, 2-hydroxyethyl methacrylate, and ethyl acetate.

In some embodiments the invention is directed to a method of applying a radiation curable nail finishing preparation including:
applying a coating of a radiation curable nail material atop a nail wherein the radiation curable nail material comprising at least a polymerizable material and a photoinitiator;
applying a base powderized coating material comprising at least a polymerizable monomer;
exposing the radiation curable material and powderized coating to a radiative source for a time suitable to cure the base materials;
applying a coating of the radiation curable nail material atop the cured materials;
applying a colored powderized coating material comprising at least a polymerizable monomer and a pigment;
exposing the radiation curable material and the colored powderized coating to a radiative source for a time suitable to cure the colored materials;
repeat the application of radiation curable material and colored powderized coating materials, and radiation curing at least once;
applying a coating of a top-coat radiation curable nail material atop the cured colored materials, and exposing the top-coat radiation curable material to a radiative source for a time suitable to cure the top-coat materials;
mechanically finish the top-coat radiation curable nail material; and
applying a second coating of the top-coat radiation curable nail material atop the mechanically finished nail surface, and exposing the top-coat radiation curable material to a radiative source for a time suitable to cure the top-coat materials.

In other embodiments, the method further includes buffing the nail prior to applying any coatings thereto.

In still other embodiments, the method further includes applying an adhesive base coat material to the surface of the nail prior to applying a coating of the radiation curable nail material.

In yet other embodiments, the nail is one of either a natural or an artificial nail.

In still yet other embodiments, the base radiation curable nail material comprises a polymerizable material comprising from 55 to 85% by weight of one or more of polyurethane acrylate oligomer and 2-hydroxyethyl methacrylate, a photoinitiator comprising from 0.2 to 2% by weight of one or more of hydroxycyclohexyl phenyl ketone and trimethylbenzoyl diphenylphosphine oxide, an inhibitor comprising less than 1% by weight of one or more of hydroquinone and p-hydroxyanisole, and from 2 to 10% by weight of at least one solvent selected from the group of butyl acetate, ethyl acetate and isopropyl alcohol.

In still yet other embodiments, the top-coat radiation curable nail material comprises a polymerizable material comprising from 5o to 85% by weight of one or more of polyurethane acrylate oligomer and 2-hydroxyethyl methacrylate, a photoinitiator comprising from 0.1 to 6% by weight of one or more of hydroxycyclohexyl phenyl ketone and trimethylbenzoyl diphenylphosphine oxide, an inhibitor comprising less than 1% by weight of one or more of hydroquinone and p-hydroxyanisole, and from 2 to 10% by weight of at least one solvent selected from the group of butyl acetate, ethyl acetate and isopropyl alcohol.

In still yet other embodiments, the powderized coating material comprises from 50 to 75% by weight of polyethyl methacrylate, from 25 to 50% by weight of polymethyl methacrylate, up to 5% by weight of dibenzoyl peroxide, up to 10% by weight of a colorant, and less than 1% by weight of a dehydrant.

Other objects, advantages and novel features, and further scope of applicability of the present invention will be set forth in part in the detailed description to follow, and in part will become apparent to those skilled in the art upon examination of the following, or may be learned by practice of the invention. The objects and advantages of the invention may be realized and attained by means of the instrumentalities and combinations particularly pointed out in the appended claims.

### BRIEF DESCRIPTION OF THE DRAWINGS

The description will be more fully understood with reference to the following FIGURES, which are presented as exemplary embodiments of the invention and should not be construed as a complete recitation of the scope of the invention, wherein:

FIG. 1 illustrates a flow chart of a method for applying a radiation compatible powder coat nail material atop a nail in accordance with embodiments of the present invention.

### DETAILED DESCRIPTION OF THE INVENTION

Turning now to the data and description, methods and processes for applying powder coat nail preparations without the need for the use of glues are provided. In many embodiments the process includes the combination of a base color gel and a powder layer, and curing that combination to build up a plurality of layers on the nail. In various other embodiments the layered gel/powder coating is finished with a top gel coat material, followed by a further curing process. In still other embodiments the process includes using a preparation material prior to applying the base gel and powder coatings. In yet other embodiments the process includes buffing and finishing the nail prior to and after applying the top gel coat material.

### Definitions

It will be understood that term “preparation” or “material”, in singular and/or plural, may substitute for composition, such as a composition of matter.

The term “(meth)acrylate” as used herein, means methacrylate, acrylate, or mixtures thereof.

The term “nail” as used herein, will be understood to mean either finger or toe nails.

The terms “powder material” or “powder coating” as used throughout the specification and claims is intended to include any powderizes acrylic material of any color.

Percentages listed in the disclosure are in weight % unless indicated otherwise.

All ranges cited in this disclosure (even where they sum to 100%) are understood to include substitutions by appropriate amounts of standard nail preparation additives, including, but not inclusive of, solvents, preservatives, coupling agents, plasticizers, secondary photoinitiators, photoaccelerators, colorants, dyes, inhibitors, oxygen scavengers, optical brighteners, dispersion aids, waxes, fillers, nanofillers, organsols, fibers, and adhesion promoting monomers or polymers, or other additives known in the art.

### Background Discussion of Conventional Powder Coat Preparations

Conventional powder coat nail preparations systems, typically incorporate three different materials:
an activated glue material;
a powder;
an activator; and
a sealer material.

While powder coat nails using these glue-based systems give adequate results, and have been extensively used, the glues are highly toxic and the removal of the glue form the nail is complicated. In particular, the time required to remove the glue-based material can take well more than an hour. This makes the use of such powder-based systems less attractive.

### Discussion of Inventive Gel-Based Powder Systems

In various embodiments the powder-based nail preparation systems are based on a combination of an a radiation curable base-gel material and gel top coat, these materials being selected to be compatible with a conventional acrylic powder coat material to allow for the preparation of the underlying nail preparation and to ensuring the gloss and sheen of the final prepared nail.

In some embodiments the powder-based nail preparation systems comprise at least a polymeric photochemical material (e.g. a gel-base coat material), a second polymeric photochemical material including an initiator (e.g., a finishing gel-base coating material), and a conventional acrylic powder based materials (e.g., powder coat material). In other embodiments, the powder-based nail preparation systems further comprises a dehydration agent to prepare the nail prior to application of the gel-base material (e.g., preparation material). In various other embodiments the systems may include suitable tools in preparing the powder coated nails, including devices for producing energetic emissions (e.g., UV and/or LED lights), and finishing tools (e.g., buffing, cutting or polishing tools).

Discussion of Gel-Nail Materials

Turning first to the gel-base materials (e.g., base and top gels). These gel-based materials in accordance with embodiments are formed from polymeric photochemical systems and/or polymeric photochemical materials, in many embodiments the photochemical material may include one or more polymerizable materials, and one or more photoinitiators and photoaccelerators, along with other conventional additives, such as fillers, preservatives, inhibitors, plasticizers, non-reactive polymers, adhesion promoters and solvents.

In some embodiments the polymeric photochemical material comprises at least a urethane oligomer, and one or more (meth)acrylated monomers, and alternatively one or more photoinitiators/accelerators, one or more inhibitors, one or more solvents and a colorant. In many embodiments, the polymerizable material may incorporate (meth)acrylate-based polymerizable monomers, as well as other polymerizable monomers, oligomers or polymers of monomers. Some embodiments of such materials include acrylates and (meth)acrylates, including, urethane(meth)acrylates, including aliphatic, aromatic, polyester, and polyether polyols and aliphatic, aromatic, polyester based polyurethanes, and polyether diisocyanates capped with (meth)acrylate endgroups. In some embodiments the polymerizable materials includes a mixture of at least a polyurethane acrylate oligomer (such as, for example, di-Hema trimethylhexyl dicarbamate), and one or more polyol methacrylates, such as, for example, 2-hydroxyethyl methacrylate (HEMA) and hydroxypropyl methacrylate.

The polymeric photochemical material may also contain one or more photoinitiators, including, for example, ketones (including benzyl ketones, sulfanyl ketones, monomeric hydroxyl ketones, polymeric hydroxyl ketones, etc.), phosphinates (including acyl phosphine oxides), metallocenes, and benzophenone and derivatives. In some embodiments, the polymerizable material may include hydroxycyclohexylphenylketone and trimethylbenzoylphosphine oxide (TPO), and mixtures thereof.

The polymeric photochemical material may also contain one or more photoaccelerators, such as for example, aliphatic amines and/or aromatic amines, such as, for example an aminobenzoate, amino (meth)acrylate.

The polymeric photochemical material may also contain a coupling agent, such as, for example, an organo-metallic such as an organo-titanate.

In addition to the above, the polymeric photochemical material may optionally include plasticizers, additional photoinitiators, colorants, solvents, dyes, preservatives, inhibitors, fillers, fibers, and/or adhesion promoting polymers. Examples of such additive materials include, but are not inclusive of: preservatives, plasticizers (e.g., phthalates, adipates, and/or sulfonamides), additional photoinitiators (e.g., quinone derivatives, dimethylketal derivatives, and or benzophenone), colorants (e.g., but not limited to, barium, calcium and aluminum lakes, iron oxides, chromates, molybdates, cadmiums, metallic or mixed metallic oxides, talcs, carmine, titanium dioxide, chromium hydroxides, ferric ferrocyanide, ultramarines, titanium dioxide coated mica platelets, and/or bismuth oxychlorides), inhibitors (e.g., hydroquinones, and/or anisoles), fillers (e.g., mineral and/or polymeric fillers), fibers, adhesion promoters (e.g., phthalates), binders (e.g., polyethylene glycol 400 dimethacrylate), and solvents (e.g., alcohols and/or acetates).

The composition ranges for some embodiments of a polymeric photochemical gel-base material is set forth in Table 1, below.

TABLE 1
Polymeric Photochemical Gel-Base Composition Ranges

| Class of | | % (by |
|---|---|---|
| Materials | Compositions | weight) |
| Polymerizable | Polyurethane acrylate oligomer | 50-75 |
| materials | 2-hydroxyethyl methacrylate | 5-10 |
| Photoinitiator/ | Hydroxycyclohexyl phenyl ketone | 0.1-1 |
| accelerators | Trimethylbenzoyl | 0.1-1 |
| | diphenylphosphine oxide | |
| Inhibitors | Hydroquinone | 0-0.005 |
| | p-hydroxyanisole | 0-0.02 |
| Colorants | (Any Suitable) | 0-0.1 (4) |
| Solvents | Butyl acetate, ethyl acetate, | 2-10 |
| | isopropyl alcohol | |

The composition ranges for some embodiments of a polymeric photochemical gel-top material is set forth in Table 2, below.

TABLE 2
Polymeric Photochemical Gel-Top Composition Ranges

| Class of | | % (by |
|---|---|---|
| Materials | Compositions | weight) |
| Polymerizable | Polyurethane acrylate oligomer | 45-75 |
| materials | 2-hydroxyethyl methacrylate | 5-10 |
| Photoinitiator/ | Hydroxycyclohexyl phenyl ketone | 0.1-1 |
| accelerators | Trimethylbenzoyl | 0-5 |
| | diphenylphosphine oxide | |
| Inhibitors | Hydroquinone | 0-0.005 |
| | p-hydroxyanisole | 0-0.02 |
| Colorants | (Any Suitable) | 0-0.1 (4) |
| Solvents | Butyl acetate, ethyl acetate, | 2-10 |
| | isopropyl alcohol | |

Regardless of the specific formulation used in making the polymeric photochemical material, the material should be curable under a radiation source, such as a UV lamp.

Turning now to the photoinitiators, preferably the radiation curable nail preparation includes at least one photoinitiator in an amount of from about 5 to 20%, preferably from about 5 to 10%. In some embodiments the photoinitiator comprises at least one photoinitiator selected from the group of phosphines, (including phosphinates and phosphine oxides), ketones (including, sulfanyl ketones and polymeric morpholinoketones), azides (including sulfonyl azides), and phosphate salts (including iodonium hexafluorophosphate), and mixtures thereof. One exemplary photoinitiator mixture is trimethylbenzoyl diphenylphosphine oxide.

Turning now to the solvents, preferably the radiation curable nail preparation includes at least one solvent in an amount of from about 1 to 10%, preferably from about 2 to 5%. In some embodiments the solvent comprises at least one solvent selected from the group of carbonates (including propylene carbonate), acetone, acetates (including ethyl and butyl acetate), toluene, and alcohols (including isopropyl alcohol).

Finally, in addition to the above, the radiation curable nail material may optionally include plasticizers, additional photoinitiators, colorants, solvents, dyes, preservatives, inhibitors, fillers, fibers, and/or adhesion promoting polymers. Examples of such additive materials include, but are not inclusive of: preservatives, plasticizers (e.g., phthalates, adipates, and/or sulfonamides), inhibitors (e.g., hydroquinones, and/or anisoles), fillers (e.g., mineral and/or polymeric fillers), fibers, and adhesion promoters (e.g., phthalates).

Again, although specific materials and formulations of radiation curable nail materials are provided above, in all cases the material is curable under exposure to a radiative source such as visible or UV light. Preferably, the composition may cure, in preferred embodiments, in less than approximately ten minutes with exposure to a radiative source, such as an actinic (UV or short wavelength source).

Discussion of Powder Coating Materials

The powder coating materials in accordance with embodiments preferable comprises at least an acrylic material and a pigment, along with solvents, preservatives, dehydrants, etc.

Exemplary acrylic materials may include, for example, one or more (meth)acrylated monomers, and alternatively one or more photoinitiators/accelerators, one or more inhibitors, one or more solvents and a colorant. In many embodiments, the polymerizable material may incorporate (meth)acrylate-based polymerizable monomers, as well as other polymerizable monomers, oligomers or polymers of monomers. Some embodiments of such materials include acrylates and (meth)acrylates, including, urethane(meth)acrylates, including aliphatic, aromatic, polyester, and polyether polyols and aliphatic, aromatic, polyester based polyurethanes, and polyether diisocyanates capped with (meth)acrylate endgroups. In some embodiments the polymerizable materials includes a mixture of at least a polyethyl and polymethyl methacrylate.

Preferably the powder coating materials also includes at least one, but potentially many, pigments including barium, calcium and aluminum lakes, iron oxides, chromates, molybdates, cadmiums, metallic or mixed metallic oxides, talcs, carmine, titanium dioxide, chromium hydroxides, ferric ferrocyanide, ultramarines, titanium dioxide coated mica platelets, and/or bismuth oxychlorides. Specific pigment formulations include D&C Black Nos. 2 & 3, FD&C Blue Nos. 1 & 4, D&C Brown No. 1, FD&C Green Nos. 3, 5, 6 & 8, D&C Orange Nos. 4, 5, 10 and 11, FD&C Red Nos. 4, 6, 7, 17, 21, 22, 27, 28, 30, 31, 33, 34, 36 and 40, D&C Violet No. 2, FD&C Yellow Nos. 5, 6, 7, 8, 10 and 11, as well as any others approved for use in cosmetics applications. Although dingle pigments are described above, it should be understood that the pigments can include mixtures of many pigments, such as for example, COLORSPERSE RED®, manufactured by Gayson Silicone Dispersions, Inc. Regardless of the ultimate pigment formulation, the pigment includes suitable preservatives and dehydration agents, including, for example, dibenzoyl peroxide and or silicon dioxide.

The composition ranges for some embodiments of a powder coating material is set forth in Table 3, below.

TABLE 3
Powder Coating Composition Ranges

| Class of | | % (by |
|---|---|---|
| Materials | Compositions | weight) |
| Polymerizable | polyethyl methacrylate | 50-75 |
| materials | polymethyl methacrylate | 25-50 |
| Initiator | dibenzoyl peroxide | 0-5 |
| Colorants | (Any Suitable) | 0-10 |
| Dehydrant | Silicon dioxide (silica) | 0-0.1 |

Regardless of the specific formulation used in making the powder coating material, the material should be compatible with a gel-based material curable under a radiation source, such as a UV lamp.

Discussion of Nail-Preparation Materials

Turning next to optional materials, in many embodiments the powder coating nail coating systems may also include a nail preparation material. These nail preparation material may include a resinous material and an acrylic material in combination with a solvent, along with other conventional additives, such as aliphatic or aromatic amines, as well as fillers, preservatives, inhibitors, plasticizers, non-reactive polymers, adhesion promoters and solvents.

In some embodiments the nail-preparation material comprises at least a resin material, such as a bisphenol A glycidyl methacrylate (bis-GMA), one or more (meth)acrylated monomers, and one or more solvents. Some embodiments of such materials include acrylates and (meth)acrylates, including, urethane(meth)acrylates, including aliphatic, aromatic, polyester, and polyether polyols and aliphatic, aromatic, polyester based polyurethanes, and polyether diisocyanates capped with (meth)acrylate endgroups. In some embodiments the polymerizable materials includes a mixture of at least a polyurethane acrylate oligomer (such as, for example, di-Hema trimethylhexyl dicarbamate), and one or more polyol methacrylates, such as, for example, 2-hydroxyethyl methacrylate (HEMA) and hydroxypropyl methacrylate. The nail-preparation materials also includes a solvent suitable for solubilizing the resins and monomers, such as, for example, ethyl acetate.

In addition to the above, the nail-preparation materials may also include plasticizers, additional photoinitiators, colorants, solvents, dyes, preservatives, inhibitors, fillers, fibers, and/or adhesion promoting polymers. Examples of such additive materials include, but are not inclusive of: preservatives, plasticizers (e.g., phthalates, adipates, and/or sulfonamides), additional photoinitiators (e.g., quinone derivatives, dimethylketal derivatives, and or benzophenone), colorants (e.g., but not limited to, barium, calcium and aluminum lakes, iron oxides, chromates, molybdates, cadmiums, metallic or mixed metallic oxides, talcs, carmine, titanium dioxide, chromium hydroxides, ferric ferrocyanide, ultramarines, titanium dioxide coated mica platelets, and/or bismuth oxychlorides), inhibitors (e.g., hydroquinones, and/or anisoles), fillers (e.g., mineral and/or polymeric fillers), fibers, adhesion promoters (e.g., phthalates), binders (e.g., polyethylene glycol 400 dimethacrylate), and solvents (e.g., alcohols and/or acetates).

The composition ranges for some embodiments of a nail preparation material is set forth in Table 4, below.

TABLE 4
Nail Preparation Composition Ranges

| Class of | | % (by |
|---|---|---|
| Materials | Compositions | weight) |
| Resin Material | 2,2-bis-(4-(2-hydroxy- | 20-30 |
| | 3-methacryloxypropoxy) | |
| | BIS-GMA | |
| Polymerizable | 2-hydroxyethyl | 20-30 |
| materials | methacrylate | |
| Solvents | ethyl acetate | 40-60 |

Regardless of the specific formulation used in making the nail preparation material, the material should be compatible with powder coating materials and gel materials curable under a radiation source, such as a UV lamp.

### Method of Applying the Powder Coat Nail Material

Although the above discussion has focused on powder coating nail materials and systems, it should be understood that other embodiments are directed to methods of applying radiation curable compatible powder coating nail materials. Unlike conventional powder coating materials these radiation curable compatible powder coating nail materials and system use base and/or top gel coat materials, the radiation curable compatible powder coating nail materials set forth in embodiments may be applied without resort to traditional glue materials, as will be discussed in greater detail below.

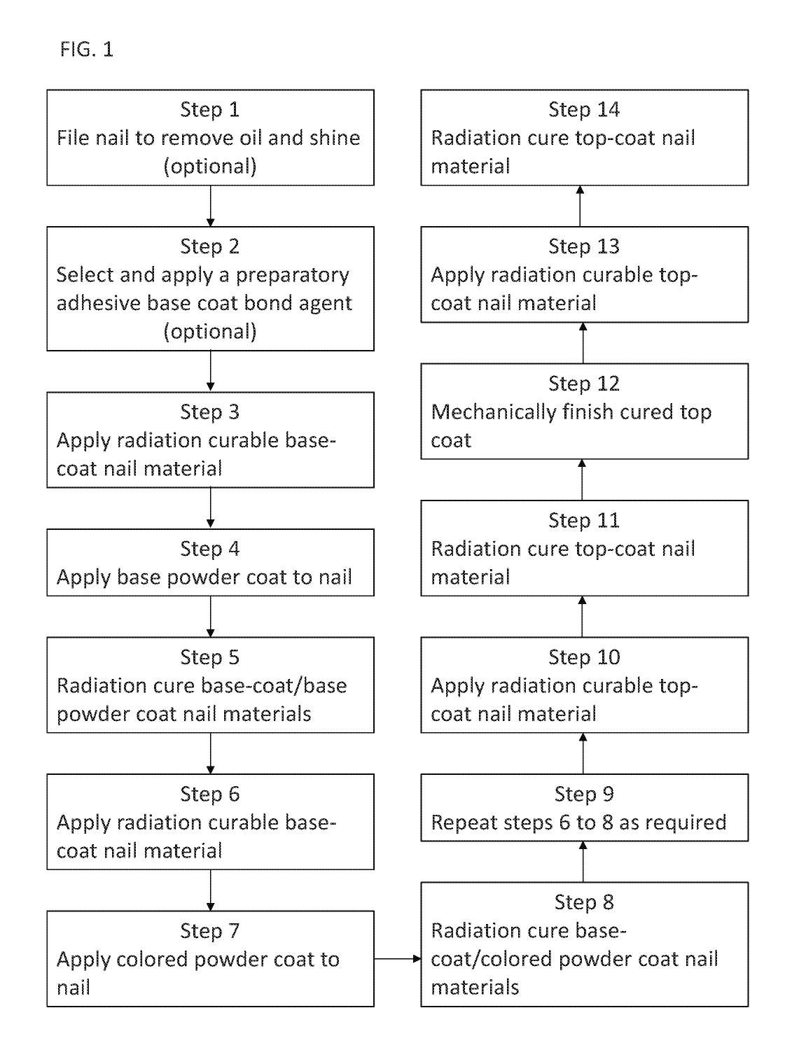

In use on either a natural nail or an artificial nail, the radiation curable compatible powder coating nail materials are applied using methods that do not require the use of conventional glue materials. In such embodiments, the nails (e.g., natural or artificial) are typically prepared by filing, and then base coats of the radiation curable gel-base and base powder coating are applied and allowed to cure, followed by the application of multiple coats of the radiation curable gel-base and a desired color power coating is/are applied and cured. After all the colored powder coat nail materials are applied, a top-gel coat material us applied and cured, then the coated nail buffed and shined, and a finale protective top-coat applied and cured. Although not necessary, it is to be understood that optionally a thin coat of a solvent-based adhesive base coat may also be applied to the surface of the nail prior to the application of the powder coating nail material. The radiation curable compatible powder coat nail material may then be applied to this preparatory adhesive base coat. The steps of such a procedure is shown in the flow chart provided in FIG. 1, and described below.

As shown in FIG. 1, some embodiments are directed to a method of creating a coated nail using a radiation curable nail material on a natural nail including the following steps:
Step 1: File nail to remove oil and shine to minimize any subsequent lifting (optional);
Step 2: Select and apply at least one preparatory coat of an adhesive base coat bond agent (optional);
Step 3: Apply one coating of a radiation curable base-coat nail material;
Step 4: Apply to the nail a base powder (optionally by dipping the nail into a powder material at a 45 degree angle);
Step 5: Expose the radiation compatible powder coat nail material to a radiative source for a time suitable to cure the material;
Step 6: Select and apply one coating of a radiation curable base-coat nail material;
Step 7: Apply to the nail a color powder (optionally by dipping the nail into a powder material at a 45 degree angle);
Step 8: Expose the radiation compatible powder coat nail material to a radiative source for a time suitable to cure the material;
Step 9; Repeat steps 6 to 8 as required;
Step 10: Apply one coating of a radiation curable top-coat nail material;
Step 11: Expose the radiation curable top-coat nail material to a radiative source for a time suitable to cure the material;
Step 12: Post-application processing, such as buffing, filing or the application of the top coating; and
Step 13: Apply one coating of a radiation curable top-coat nail material; and
Step 14: Expose the radiation curable top-coat nail material to a radiative source for a time suitable to cure the material.

As provided in the flowchart of FIG. 1, in many embodiments, the radiation compatible powder coat nail material may be applied to any sort of nail. In such embodiment, the nail may be optionally filed on the top surface to remove oils and create a surface for bonding. Again, although not necessary for the operation of the radiation compatible powder coat nail materials of the disclosure, atop this untreated nail an adhesive preparatory base coat material may also optionally be applied to part or the entire surface of the nail. Once any optional preparatory steps are completed a radiation curable nail material is applied followed by a base powder coat material to the nail and cured via exposure to a radiative source. Once the base coat is cured, the radiation curable nail material is applied followed by a color powder coat material to the nail and cured via exposure to a radiative source The application of multiple such coatings or partial colored coatings of the same or different radiation compatible powder coat nail materials may be made to create the final nail effect desired. For example, if a French manicure look is desired a first white coat or white tip coat of radiation curable nail material may be applied and cured, followed by a clear or colored coating of such material, which would then be cured. The coated nail is then coated with a clear radiation-curable top coating material and the top coating cured. The top-coated nail is then filed and buffed and at least a second coat of the clear radiation-curable top coating material is applied and the top coating cured. Although one specific method is described, other colors of coatings are anticipated and may be used in any number of combinations or alone.

### DOCTRINE OF EQUIVALENTS

Although the invention has been described in detail with particular reference to these preferred embodiments, other embodiments can achieve the same results. Variations and modifications of the present invention will be obvious to those skilled in the art and it is intended to cover all such modifications and equivalents. The entire disclosures of all references, applications, patents, and publications cited above, and of the corresponding application(s), are hereby incorporated by reference.

What is claimed is:

1. A radiation compatible powder nail coating system comprising:
a base radiation curable nail material comprising at least a polymerizable material and a photoinitiator;
at least one base and one colored powderized coating material, each comprising at least a polymerizable monomer, and one of which further comprising a pigment material;
a top-coat radiation curable nail material comprising a polymerizable material and a photoinitiator; and
a radiative source.

2. The radiation compatible powder nail coating system of claim 1, further comprising a solvent-based resin preparation material.

3. The radiation compatible nail coating system of claim 1, wherein the base radiation curable nail material and the top-coat radiation curable material comprise at least one material selected from the group consisting of acrylates, (meth)acrylates, urethane(meth)acrylates, aliphatic polyols, aromatic polyols, polyester polyols, polyether polyols, aliphatic polyurethanes, aromatic polyurethanes, polyester polyurethanes, polyether diisocyanates, di-Hema trimethylhexyl dicarbamate), 2-hydroxyethyl methacrylate, hydroxypropyl methacrylate, and mixtures thereof.

4. The radiation compatible nail coating system of claim 1, wherein the base radiation curable nail material and the top-coat radiation curable material comprise at least one material selected from the group consisting of ketones, benzyl ketones, sulfanyl ketones, monomeric hydroxyl ketones, polymeric hydroxyl ketones, phosphinates, acyl phosphine oxides, metallocenes, benzophenone, hydroxycyclohexylphenylketone, trimethylbenzoylphosphine oxid), and mixtures thereof.

5. The radiation compatible coating system of claim 1, further comprising a material selected from the group of photoaccelerators, coupling agents, plasticizers, additional photoinitiators, colorants, solvents, dyes, preservatives, inhibitors, fillers, fibers, and adhesion promoting polymers

6. The radiation compatible coating system of claim 1, wherein the base radiation curable nail material comprises from 55 to 85% by weight of one or more polymerizable material, 0.2 to 2% by weight of one or more photoinitiator, one or more inhibitor, and from 2 to 10% by weight of one or more solvent.

7. The radiation compatible coating system of claim 6, wherein the polymerizable material comprises a mixture of polyurethane acrylate oligomer and 2-hydroxyethyl methacrylate, the photoinitator comprises one or more of hydroxycyclohexyl phenyl ketone and trimethylbenzoyl diphenylphosphine oxide, the inhibitor comprises one or more of hydroquinone and p-hydroxyanisole, and wherein the solvent is one or more of butyl acetate, ethyl acetate and isopropyl alcohol.

8. The radiation compatible coating system of claim 1, wherein the top-coat radiation curable material comprises from 50 to 85% by weight of one or more polymerizable materials, 0.1 to 6% by weight of one or more photoinitiator, one or more inhibitor, and from 2 to 10% by weight of one or more solvent.

9. The radiation compatible coating system of claim 1, wherein the powderized coating materials comprise one or more materials selected from the group of acrylates, (meth)acrylates, urethane(meth)acrylates, and polyether diisocyanates.

10. The radiation compatible coating system of claim 1, further comprising a preparatory base coat comprising a resin material, a polymerizable material and a solvent.

11. The radiation compatible coating system of claim 10, wherein the resin material comprises from 20 to 30% by weight, the polymerizable material comprises from 20 to 30% by weight, and wherein the solvent comprises from 40 to 60% by weight.

12. The radiation compatible coating system of claim 10, wherein the preparatory base coat comprises one or more material selected from the group consisting of bisphenol A glycidyl methacrylate (bis-GMA), (meth)acrylated monomers, urethane(meth)acrylates, polyurethanes, polyether diisocyanates, di-Hema trimethylhexyl dicarbamate), 2-hydroxyethyl methacrylate (HEMA) and hydroxypropyl methacrylate.

13. The radiation compatible coating system of claim 10, wherein the preparatory base coat comprises 2,2-bis-(4-(2-hydroxy-3-methacryloxypropoxy)BIS-GMA, 2-hydroxyethyl methacrylate, and ethyl acetate.

14. A method of applying a radiation curable nail finishing preparation comprising:
applying a coating of a radiation curable nail material atop a nail wherein the radiation curable nail material comprising at least a polymerizable material and a photoinitiator;
applying a base powderized coating material comprising at least a polymerizable monomer;
exposing the radiation curable material and powderized coating to a radiative source for a time suitable to cure the base materials;
applying a coating of the radiation curable nail material atop the cured materials;
applying a colored powderized coating material comprising at least a polymerizable monomer and a pigment;
exposing the radiation curable material and the colored powderized coating to a radiative source for a time suitable to cure the colored materials;
repeat the application of radiation curable material and colored powderized coating materials, and radiation curing at least once;
applying a coating of a top-coat radiation curable nail material atop the cured colored materials, and exposing the top-coat radiation curable material to a radiative source for a time suitable to cure the top-coat materials;
mechanically finish the top-coat radiation curable nail material; and
applying a second coating of the top-coat radiation curable nail material atop the mechanically finished nail surface, and exposing the top-coat radiation curable material to a radiative source for a time suitable to cure the top-coat materials.

15. The method of claim 14, further comprising buffing the nail prior to applying any coatings thereto.

16. The method of claim 14, further comprising applying an adhesive base coat material to the surface of the nail prior to applying a coating of the radiation curable nail material.

17. The method of claim 14, wherein the nail is one of either a natural or an artificial nail.

18. The method of claim 14, wherein the base radiation curable nail material comprises a polymerizable material comprising from 55 to 85% by weight of one or more of polyurethane acrylate oligomer and 2-hydroxyethyl methacrylate, a photoinitiator comprising from 0.2 to 2% by weight of one or more of hydroxycyclohexyl phenyl ketone and trimethylbenzoyl diphenylphosphine oxide, an inhibitor comprising less than 1% by weight of one or more of hydroquinone and p-hydroxyanisole, and from 2 to 10% by weight of at least one solvent selected from the group of butyl acetate, ethyl acetate and isopropyl alcohol.

19. The method of claim 14, wherein the top-coat radiation curable nail material comprises a polymerizable material comprising from 5o to 85% by weight of one or more of polyurethane acrylate oligomer and 2-hydroxyethyl methacrylate, a photoinitiator comprising from 0.1 to 6% by weight of one or more of hydroxycyclohexyl phenyl ketone and trimethylbenzoyl diphenylphosphine oxide, an inhibitor comprising less than 1% by weight of one or more of hydroquinone and p-hydroxyanisole, and from 2 to 10% by weight of at least one solvent selected from the group of butyl acetate, ethyl acetate and isopropyl alcohol.

20. The method of claim 14, wherein the powderized coating material comprises from 50 to 75% by weight of polyethyl methacrylate, from 25 to 50% by weight of polymethyl methacrylate, up to 5% by weight of dibenzoyl peroxide, up to 10% by weight of a colorant, and less than 1% by weight of a dehydrant.
